(19) United States
(12) Patent Application Publication
(10) Pub. No.: US 2006/0024285 A1
(43) Pub. Date: Feb. 2, 2006

(54) Carcinogen detoxification composition and method

(75) Inventor: Yi-Ming A. Chen, Taipei (TW)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Gene Research Lab. Co., Ltd., Taipei (TW)

(21) Appl. No.: 11/193,205

(22) Filed: Jul. 29, 2005

Related U.S. Application Data
(60) Provisional application No. 60/600,367, filed on Aug. 11, 2004.

(30) Foreign Application Priority Data
Jul. 30, 2004 (EP) 04 018 113.3

(51) Int. Cl. A61K 38/48; A61K 39/395

(52) U.S. Cl. 424/94.5; 424/130.1

(57) ABSTRACT

A pharmaceutical, food or cosmetic composition comprising a carrier and an effective amount of an active benzo(a)pyrene binding protein, whereby the protein is a SAM-dependent methyltransferase or a function-conservative variant or fragment thereof, having a SAM-binding domain specifically binding benzo(a)pyrene.

### CROSS-REFERENCE TO RELATED APPLICATIONS

This application claims the benefit of European Patent Application No. 04 018 113.3, filed Jul. 30, 2004 and U.S. Provisional Application No. 60/600,367, filed Aug. 11, 2004; both of which are hereby incorporated herein in their entirety by reference.

### FIELD OF THE INVENTION

The present invention relates to a pharmaceutical, food or cosmetic composition containing proteins capable of binding specific carcinogens in vivo. More specifically, the present invention relates to a pharmaceutical, food or cosmetic composition containing proteins capable of binding benzo(a)pyrene in vivo. Moreover, the present invention relates to the use of the proteins for the prevention or treatment of cancer. The present invention also relates compositions for use in medicine, which contain the proteins of the invention.

### BACKGROUND OF THE INVENTION

The benzo(a)pyrene (BaP) is a carcinogen having the following formula.

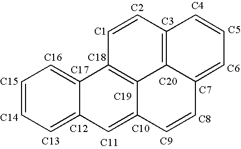

BaP is generated by combustion of organic material, Workers in gas generation and steel plants, and individuals engaged in aluminum reduction and roofing have higher cancer risks associated with long-term exposure to various polycyclic aromatic hydrocarbons. (PAHs) including BaP (1). After diffusing into a cell, BaP binds at an aryl hydrocarbon receptor (AhR), translocates into the cell's nucleus, and transactivates the CYP1A1 gene (24). A metabolic BaP product known as BaP 7,8-dihydrodiol-9,10-epoxide (BPDE) is capable of forming DNA adducts and triggering mutagenesis (5).

Glycine N-methyltransferase (GNMT, EC2.1.1.20), a protein with multiple functions, affects genetic stability by a) regulating the ratio of SAM to S-adenosylhomocystine (SAH) and b) binding to folate (6, 7). The present inventors have previously reported on diminished GNMT expression levels in both human hepatocellular carcinoma (HCC) cell lines and tumorous tissues (8, 9). In previous projects, the human GNMT gene was localized to the 6p12 chromosomal region and its polymorphism was characterized (10, 11). Genotypic analyses of several human GNMT gene polymorphisms showed a loss of heterozygocity in 36-47% of the genetic markers in HCC tissues (11).

### SUMMARY OF THE INVENTION

It is a problem of the present invention to provide pharmaceutical, food or cosmetic compositions useful for the prevention and treatment of cancer, notably hepatoma, lung cancer, bladder cancer, prostate cancer, colon cancer, brain tumor, breast cancer, and kidney cancer of mammals including humans.

It is a further problem of the invention to provide a novel use for GNMT as a medical treatment for the human or animal body.

It is a still further problem of the present invention to provide a method for the prevention or treatment of BaP mediated carcinogenesis, in particular hepatoma, lung cancer, bladder cancer, prostate cancer, colon cancer, brain tumor, breast cancer, and kidney cancer of mammals including humans.

These problems are solved according to the claims by a pharmaceutical, food or cosmetic composition comprising a carrier and an effective amount of an active benzo(a)pyrene binding protein, whereby the protein is a SAM-dependent methyltransferase or a function-conservative variant or fragment thereof, having a SAM-binding domain specifically binding benzo(a)pyrene. The methyltransferase in a composition according to the invention is preferably GNMT, HhaI-DNA MTases, HaeIII-DNA MTases or PvuII-DNA MTases. Most preferably, the methyltransferase is GNMT (Chen Y M, Chen L Y, Wong F H, Lee C M; Chang T J, Yang-Feng T L. Genomics. 2000 May 15; 66(1):43-7. PMID: 10843803 [PubMed—indexed for MEDLINE]), which has the following amino acid sequence:

| SEQ ID No: 1 | | |
|---|---|---|
| 1- | MVDSVYRTRSLGVAAEGLPDQYADGEAARVWQLYIGDTRSRTAEYKAWLL | -50 |
| 51- | GLLRQHGCQRVLDVACGTGVDSIMLVEEGFSVTSVDASDKMLKYALKERW | -100 |
| 101- | NRRHEPAPDKWVIEEANWMTLDKDVPQSAEGGPDAVICLGNSFAHLPDCK | -150 |
| 151- | GDQSEHRLALKNIASMVRAGGLLVIDHRNYDHILSTGCAPPGKNIYYKSD | -200 |
| 201- | LTKDVTTSVLIVNNKAHMVTLDYTVQVPGAGQDGSPGLSKFRLSYYPHCL | -250 |
| 251- | ASPTELLQAAFGGKCQHSVLGDFKPYKPGQTYIPCYFIHVLKRTD | -295 |

GNMT sequence data have been deposited with the EMBL/GenBank Data libraries under Accession No. AF101475.

The present invention is based on the recognition that GNMT as an element of a specific subclass of methyl transferases is involved in a novel detoxification pathway of the carcinogen BaP. Specifically, the present invention is based on the recognition of a BaP binding preference in vivo for the SAM-binding domain of GNMT and other SAM-dependent methyltransferases (MTases) indicating that BaP readily interacts with DNA methyl transferases that use cytosine as a target moiety: When GNMT-overexpressing transgenic mice are treated with B(a)P, only 30% of the mice generated lung tumors whereas normal mice lacking GNMT over expression generate a lung tumor at a rate of 67% under the same conditions. Accordingly, GNMT binding of B(a)P in vivo is capable of preventing carcinogenesis.

The present invention further provides the use of a SAM-dependent methyltransferase or a function-conservative variant or fragment thereof, having a SAM-binding domain specifically binding benzo(a)pyrene for the manufacture of a medicament for the prevention or treatment of cancer, in particular hepatoma, lung cancer, bladder cancer, prostate cancer, colon cancer, brain tumor, breast cancer, and kidney cancer of mammals including humans. The composition may be administered orally, topically or parenterally. Preferably, the methyltransferase is GNMT, HhaI-DNA MTases, HaeIII-DNA MTases or PvuII-DNA MTases. Most preferably, the methyltransferase is GNMT.

The present invention also provides a method for the prevention or treatment of cancer which comprises administering a pharmaceutically effective amount of an SAM-dependent methyltransferase or a function-conservative variant or fragment thereof, having a SAM-binding domain specifically binding benzo(a)pyrene, to an individual. The SAM-dependent methyltransferase or a function-conservative variant or fragment thereof, having a SAM-binding domain specifically binding benzo(a)pyrene GNMT may be directly administered or by way of a vector encoding for the protein, whereby the vector is capable of expressing the protein in vivo.

### DETAILED DESCRIPTION OF THE INVENTION

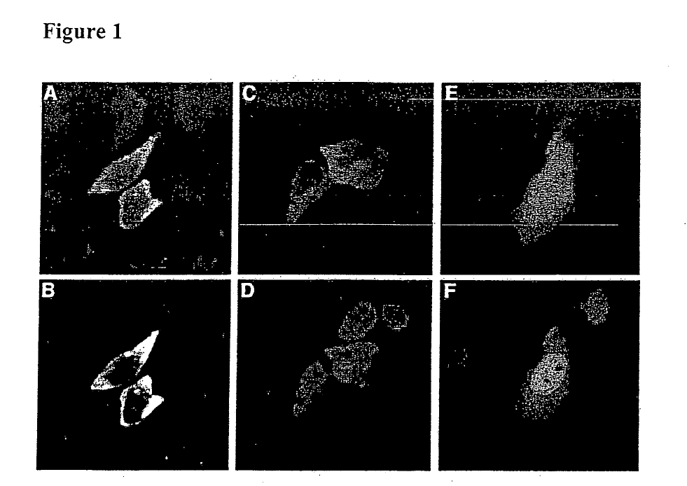

FIG. 1. Nuclear translocation of GNMT following cell treatment with BaP. Photos A and B: a double IFA was performed on HA22T/VGH cells transfected with pGNMT. Antisera: A, rabbit anti-GNMT antibody; B, mouse anti-Flag antiserum. Photos C-F: IFA on Huh 7 cells transfected with pGNMT and treated with either DMSO solvent (C and D) or BaP (E and F) prior to being fixed and reacted with mouse anti-Flag antiserum. Immunofluorescent staining was performed with Rhodamine-conjugated goat anti-rabbit antibodies (A) or FITC-conjugated rabbit anti-mouse antibodies (B-F). Nuclei were stained with Hoechst H33258.

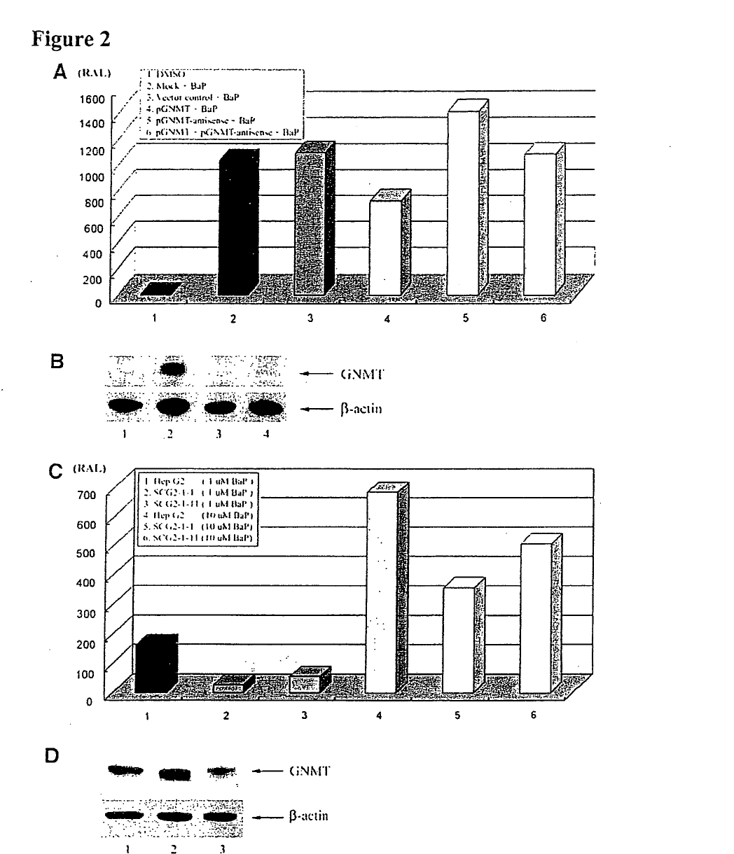

FIG. 2. Effects of GNMT on BPDE-DNA adduct formation. (A) Amount (RAL) of BPDE-DNA adducts using a combination of ³²P-postlabeling and 5 dimensional thin-layer chromatography. Lane 1, DMSO solvent control; lane 2, mock; lane 3, cells transfected with 40 μg control (pFLAG-CMV-5) vector; lane 4, cells transfected with 40 μg pGNMT; lane 5, cells transfected with 40 μg pGNMT-antisense; lane 6, cells co-transfected with 20 μg pGNMT and 20 μg pGNMT-antisense. DNA adduct quantities per 10⁸ nucleotides (relative adducts level, RAL): lane 1, 0; lane 2, 1031.7; lane 3, 1092.4; lane 4, 719.8; lane 5, 1411.3; lane 6, 1079.7. (B) Western blot analysis of GNMT expression in Hep G2 cells transfected with the control (pFLAG-CMV-5) vector (lane 1), pGNMT (lane 2), pGNMT-antisense (lane 3), or pGNMT/pGNMT-antisense (lane 4). Bottom row shows β-actin expression levels for the four experiments. (C) Amounts of BPDE-DNA adducts in Hep G2, SCG2-1-1, and SCG2-1-11 cells treated with 1 or 10 μM BaP. Lanes 1 and 4: Hep G2 cells treated, with 1 or 10 μM Bap; lanes 2 and 5: SCG2-1-1 treated with 1 or 10 μM BaP; lanes 3 and 6: SCG2-1-11 treated with 1 or 10 μM BaP. DNA adducts quantities per 10⁸ nucleotides (RAL): lane 1, 161.9; lane 2, 26.4; lane 3, 55.2; lane 4, 682.1; lane 5, 354.9; lane 6, 506.5. (D) Western blot analysis of GNMT expression in Hep G2 (lane 1), SCG2-1-1 (lane 2) and SCG2-1-11 (lane 3) cells. Twenty μg cell lysates from each cell line were used for the polyacrylamide gel-electrophoresis. Bottom row shows β-actin expression levels for the four experiments.

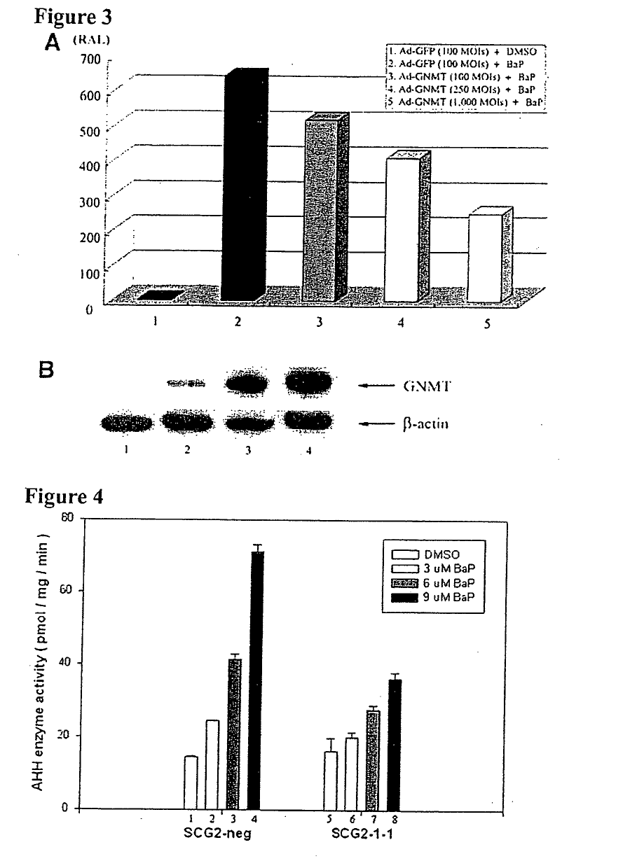

FIG. 3. Effects of GNMT expression on BPDE-DNA adduct formation in Hep G2 cells infected with Ad-GFP or various MOIs of Ad-GNMT. (A) lane 1, cells infected with Ad-GFP and treated with DMSO solvent; lane 2, cells infected with Ad-GFP and treated with BaP; lane 3, cells infected with 100 MOIs of Ad-GNMT and treated with BaP; lane 4, cells infected with 250 MOIs of Ad-GNMT and treated with BaP: lane 5, cells infected with 1,000 MOIs of Ad-GNMT and treated with BaP. DNA adduct quantities per 10⁸ nucleotides (relative adducts level, RAL): lane 1, 0; lane 2, 638.9; lane 3, 514.2; lane 4, 405.3; lane 5, 224.3. (B) Western blot analysis of GNMT expression in the same experiment. Lane 1, Ad-GFP control; lane 2, Ad-GNMT (100 MOIs); lane 3, Ad-GNMT (250 MOIs); lane 4, Ad-GNMT (1,000 MOIs).

FIG. 4. Cytochrome p450 1A1 (CYP1A1) enzyme activity induced by BaP in SCG2-neg and SCG2-1-1 cells as measured by an aryl hydrocarbon hydroxylase (AHH) assay. Lanes 1-4, CYP1A1 activity in SCG2-neg; lanes 5-8, in SCG2-1-1. Treatments: lanes 1 and 5, DMSO solvent; lanes 2 and 6, 3 μM BaP; lanes 3 and 7, 6 μM BaP; lanes 4 and 8, 9 μM BaP. The CYP1A1 enzyme activity, means (pmol/mg/min) and standard deviations (in parentheses): lane 1, 14.5 (0.27); lane 2, 24.47 (0.14); lane 3, 41.5 (1.42); lane 4, 71.3 (1.75); lane 5, 16.2 (3.6); lane 6, 20.1 (1.5); lane 7, 27.7 (1.2); lane 8, 36.2 (1.7).

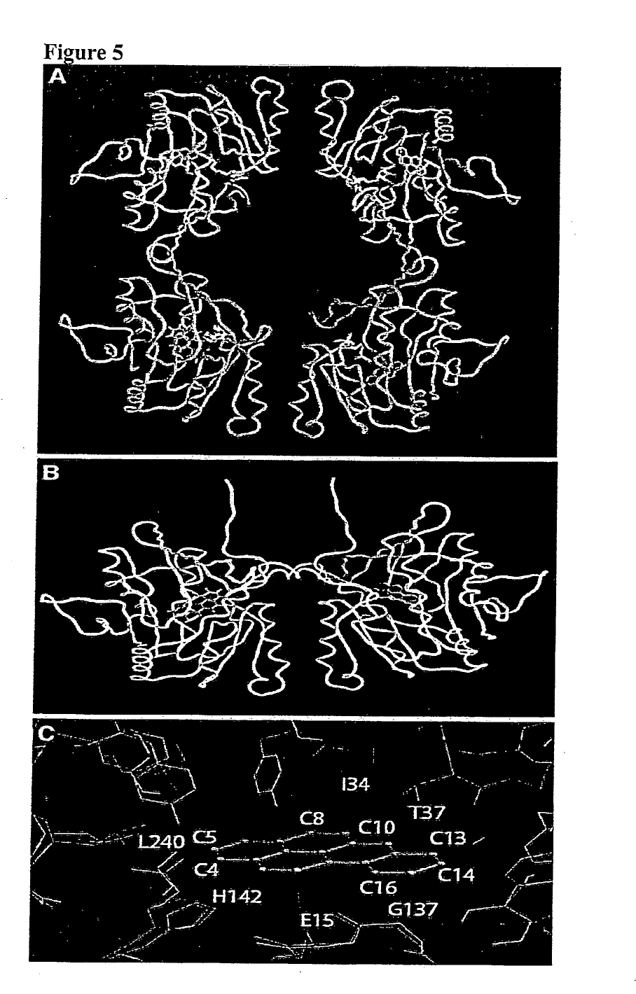

FIG. 5. Model of BaP docking with dimeric and tetrameric forms of GNMT using the Lamarckian genetic algorithm. (A) BaP (red) docked with SAH (white) bound tetrameric form of rat GNMT (cyan, 1 D2H). (B) BaP (red) docked with the dimeric form of rat GNMT (yellow, 1D2C). (C) Dimeric form of GNMT (yellow) superimposed on tetrameric form of GNMT (cyan). GNMT amino acid residues (Ile34, Thr37, Gly137, His142 and Leu240 of one dimeric subunit and Glu15 of another) in close proximity to several BaP carbon atoms are indicated based on the 1D2C and BaP docking model.

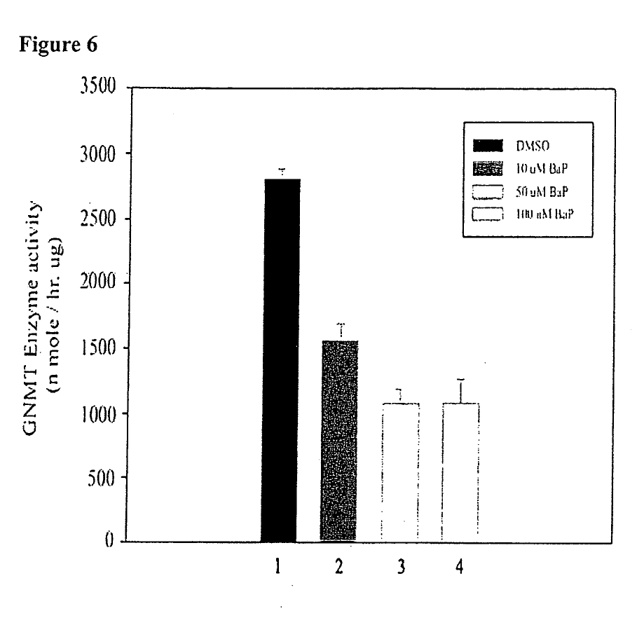

FIG. 6. Inhibition of GNMT enzyme activity by BaP. GNMT enzyme activity was measured as 2810.8±73.7 nmol/hr/μg for treatment with DMSO solvent; 1563.3±127.4 nmol/hr/μg for treatment with 10 μM BaP; 1069.5±124.2 10 μM for treatment with 50 μM BaP; and 1083.3±175.9 nmol/hr/μg for treatment with 100 μM BaP. Each reaction set was performed in triplicate, as were individual experiments.

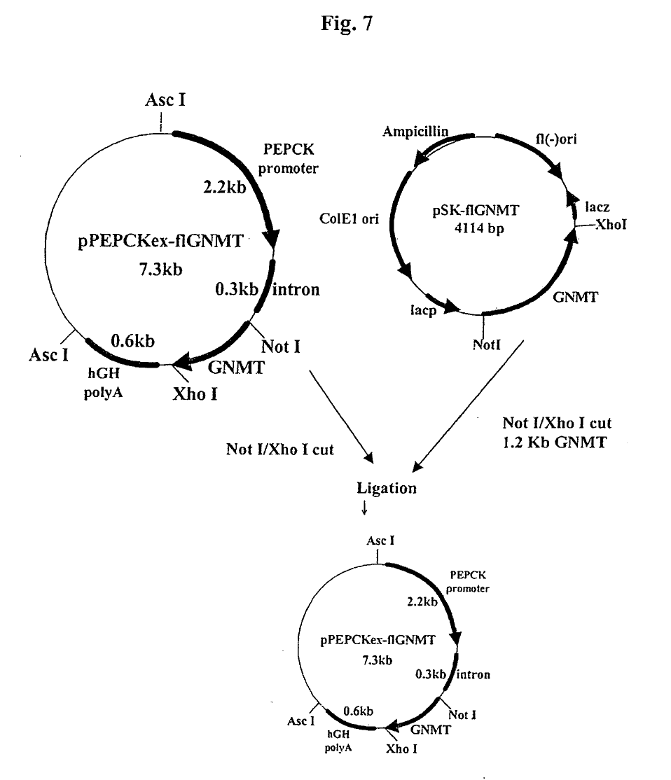

FIG. 7 Construct of the pPEPCKex-flGNMT plasmid. pPEPCKex (vector) and pSK-flGNMT (insert) were digested with Not I and Xho I and ligated to generate pPEPCKex-flGNMT.

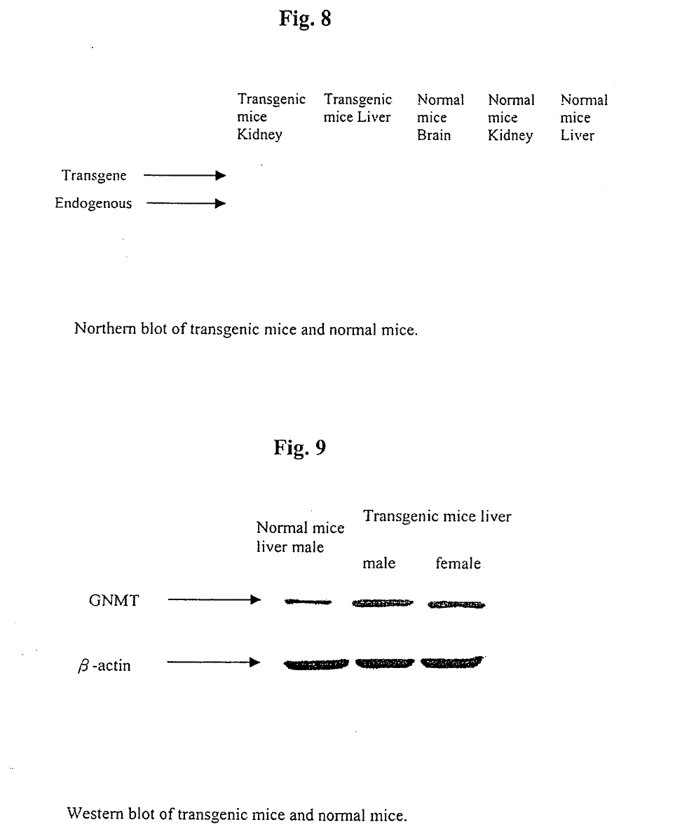

FIG. 8 Northern blot of transgenic mice and normal mice.

FIG. 9 Western blot of transgenic mice and normal mice.

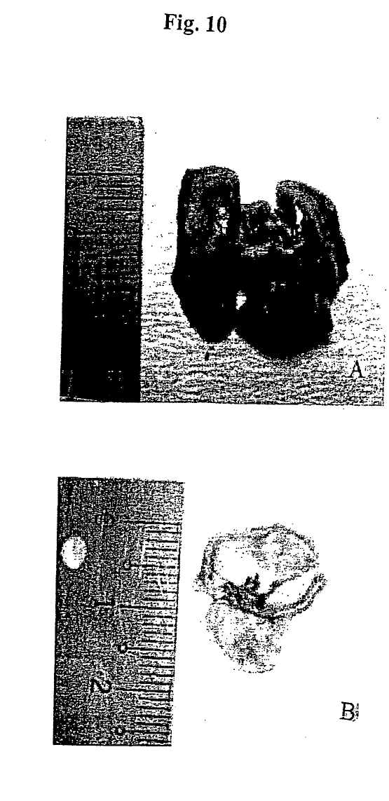

FIG. 10 Pathology of the lung organs of GNMT transgenic mice (A) and normal mice (B) treated with BaP and sacrificed 78 weeks after the challenge.

The present invention compositions and methods for preventing and creating disease conditions in humans associated with BaP induced carcinogenis. Therapeutic and prophylactic compositions of the invention comprise at least one SAM-dependent methyltransferase or a function-conservative variant or fragment thereof, having a SAM-binding domain specifically binding benzo(a)pyrene. The methyltransferase protein contained in the composition of the invention may be an isolated, purified protein, essentially free of all other proteins or contaminants. The methyltransferase protein may also be contained in the composition of the invention in the form of a mixture obtained from a natural source, e.g. as an extract. If the composition contains a mixture obtained from a natural source, then the composition of the invention contains the methyltransferase protein in a concentration which is higher than the concentration of the methyltransferase in the natural source. Preferaby, the concentration of the methyltransferase is contained in a concentration which is at least 2 times, more preferably 3 to 1000 times, higher than the concentration of the methyltransferase in the natural source.

A composition according to the invention is capable of treating or preventing carcinogenesis when administered to a patient in a therapeutic regimen. Compositions and methods according to the invention may be used to treat disease conditions related to benzo(a)pyrene (BaP) carcinogens and derivatives thereof. In vivo tests described in the Examples demonstrate the successful use of GNMT as an element of a specific subclass of methyl transferases, in the prevention and treatment of carcinogenisis. The subclass is characterized by an SAM binding domain which at the same time selectively binds BaP.

In accordance with this invention, a “protein” refers to a defined sequence of amino acid residues preferably comprising no more than about 1000 amino acid residues and comprising at least approximately 50 amino acid residues in length, and preferably at least about 100 amino acid residues in length, and more preferably at least about 150 amino acid residues in length and which, when derived from a methyl transferase, contains the same number of amino acid residues or less than the amino acid sequence of the entire methyl transferase and in a particular embodiment no more than about 95% of the amino acid residues of the entire protein, but including an effective SAM binding domain. Proteins used in accordance with the invention comprise at least one SAM binding domain. A SAM binding domain is the basic element or smallest unit of recognition of BaP and necessary for binding BaP in vivo. The SAM binding domains are believed to be involved in binding BaP in vivo thereby avoiding the diffusion of BaP into a cell, binding with an aryl hydrocarbon receptor (AhR), translocation into the cell's nucleus, or transactivation of the CYP1A1 gene. Accordingly, the SAM-dependent methyltransferase or function-conservative variant or fragment thereof, having a SAM-binding domain specifically binding benzo(a)pyrene are useful in the prevention or treatment of carcinogenisis. The most preferred protein according to the invention is GNMT. The contact distances between GNMT (pdb:1D2C) and BaP based on a docking model are shown in Reference Table 1 below in order to illustrate a binding pocket of GNMT.

REFERENCE TABLE 1

| GNMT . . . BaP | (Contact) | Distance (A) |
|---|---|---|
| A19(Met)CE | C6 | 3.72 |
| A37(Thr)OG1 | C11 | 3.38 |
| A37(Thr)OG2 | C9 | 3.43 |
| A137(Gly)O | C16 | 3.05 |
| A137(Gly)O | C1 | 3.38 |
| A142(His)NE2 | C4 | 3.22 |
| A142(His)NE2 | C2 | 3.40 |
| A191(Asn)ND2 | C14 | 3.24 |
| A283(Tyr)OH | C15 | 3.74 |
| B15(Glu)OE2 | C7 | 3.38 |
| B15(Glu)OE1 | C7 | 3.58 |

A therapeutic/prophylactic treatment regimen in accordance with the invention (which results in prevention of, or delay in, the onset of disease symptoms caused by BaP) comprises administration of a composition of the invention comprising at least one SAM-dependent methyltransferase or function-conservative variant or fragment thereof, having a SAM-binding domain specifically binding benzo(a)pyrene. The use of a composition of the invention may:
(a) bind BaP or a derivative thereof present in solid or liquid food or fluids such as smoke or vapors, to which an individual is exposed prior to resorption of BaP into the body of the individual,
(b) bind BaP or a derivative in the body of the individual.

Compositions and methods of the invention are useful for treating cancer, such as hepatoma, lung cancer, bladder cancer, prostate cancer, colon cancer, brain tumor, breast cancer, and kidney cancer in mammals including humans.

Proteins having a defined sequence of amino acid residues comprising at least one SAM binding domain specifically binding benzo(a)pyrene of a SAM-dependent methyltransferase or a function-conservative variant thereof may contain the amino acid sequence of known methyltransferases, such as GNMT having an amino acid sequence as shown in SEQ. ID. No.: 1.

In addition, proteins having defined amino acid compositions and which comprise at least one SAM binding domain specifically binding benzo(a)pyrene of a SAM-dependent methyltransferase or a function-conservative fragment or variant thereof may be identified for any known methyl transferase, including GNMT. One method directed to the provision of function-conservative fragments includes dividing the protein into non-overlapping, or overlapping peptides of desired lengths and synthesizing, purifying and testing those peptides to determine whether the peptides comprise at least one SAM binding domain specifically binding benzo(a)pyrene and derivatives thereof. In another method, an algorithm is used for predicting those peptides which are likely to comprise a SAM binding domain specifically binding benzo(a)pyrene, and then synthesizing, purifying and testing the peptides predicted by the algorithm in cell assays, e.g. as described in the present examples, to determine if such predicted peptides specifically bind to BaP. Preferably, a protein has equal or higher binding capability to BaP as compared with GNMT. Preferred protein fragments useful in accordance with this invention comprise at least one SAM binding domain specifically binding benzo(a)pyrene.

It is also possible to modify the structure of any of the above-described proteins for use as a function-conservative variant in accordance with the present invention for such purposes as increasing solubility (particularly desirable if the composition is to be injected), enhancing therapeutic or preventive efficacy, or stability (e.g., shelf life ex vivo, and resistance to proteolytic degradation in vivo). A function-conservative variant can be produced in which the amino acid sequence has been altered as compared to the native protein sequence from which it is derived, or as compared to the protein fragment to be modified such as by amino acid substitution, deletion, or addition, to modify BaP binding capability, or to which a component has been added for the same purpose.

A composition according to the invention may be prepared based on a mixture containing a methyltransferase protein from a natural source. The mixture containing the methyltransferase protein from a natural source may be obtained by any suitable method such as extraction of a suitable starting material. A suitable natural source may be based on microorganisms or animals. For the purposes of the present invention it is not essential that the methyl transferase is isolated in pure form provided that the methyl transferase contained in the mixture is active in binding BaP. Accordingly, it is possible to use the mixture as such as long as the methyl transferase is present in a concentration sufficient to provide the necessary activity.

A mixture containing GNMT according to the invention may be based on a microorganism, in particular a yeast, or a mixture extracted from a microorganism.

A mixture containing GNMT according to the invention may be based on an organ of an animal. A suitable animal may be selected from pigs, cattle, or rabbit. A suitable organ of an animal may be selected from liver, pancreas or prostate.

The proteins of the invention may be obtained as an extract from a natural source by using standard means or methods, such as by contacting the material with an appropriate solvent to prepare a tincture, or by any other conventional means or method, such as by carbon dioxide extraction, freeze-drying, or spray-drying (See Gennaro A R: Remington: The Science and Practice of Pharmacy, Mack Publishing Company, Easton Pa. 1995 and The United States Pharmacopeia 22nd rev, and The National Formulary (NF) 17 ed, USP Convention, Rockville Md., 1990.)

The extract is prepared using a microorganism or a homogeneate thereof, an animal organ or a homogeneate thereof, all containing proteins of the invention, and a solvent, which may be water, such as distilled water, an aqueous solvent, such as PBS, saline or water combined with other solvents, an organic solvent, such as DMSO, DMF, or an alcohol, such as ethanol or isopropanol, or any combination thereof. The resulting extract is typically composed of a wet or liquid component and a solid component.

Highly purified peptides free from all other polypeptides and contaminants having a defined sequence of amino acid residues comprising at least one SAM binding domain specifically binding benzo(a)pyrene, may be produced synthetically by chemical synthesis using standard techniques. Various methods of chemically synthesizing peptides are known in the art such as solid phase synthesis whereby the protein is anchored to a polymer support (solid phase synthesis) or by conventional homogenous chemical reactions (solution synthesis). Synthetically produced peptides may then be purified to homogeneity (i.e. at least 90%, more preferably at least 95% and even more preferably at least 97% purity), optionally free from all other polypeptides and contaminants using techniques known in the literature for protein purification.

In accordance with one embodiment for producing highly purified homogenous peptide compositions, a protein produced by synthetic chemical means may be purified by preparative reverse phase chromatography. In this method, the synthetically produced peptide in crude form is dissolved in an appropriate solvent (typically an aqueous buffer) and applied to a separation column (typically a reverse phase silica based media, in addition, polymer or carbon based media may be used). Peptide is eluted from the column by increasing the concentration of an organic component (typically acetonitrile or methanol) in an aqueous buffer (typically TFA, triethylamine phosphate, acetate or similar buffer). Fractions of the eluate will be collected and analyzed by appropriate analytical methods (typically reverse phase HPLC or CZE chromatography). Those fractions having the required homogeneity will be pooled. The counter ion present may be changed by additional reverse phase chromatography in the salt of choice or by ion exchange resins. The peptide may then be isolated as its acetate or other appropriate salt. The peptide is then filtered and the water removed (typically by lyophilization) to give a homogenous peptide composition containing at least 90%, more preferably at least 95% and even more preferably at least 97% of the required peptide component. Optionally, or in conjunction with reverse phase HPLC as described above, purification may be accomplished by affinity chromatography, ion exchange, size exclusion, counter current or normal phase separation systems, or any combination of these methods. Peptide may additionally be concentrated using ultra filtration, rotary evaporation, precipitation, dialysis or other similar techniques.

The highly purified homogenous peptide composition may be characterized by any of the following techniques or combinations thereof: a) mass spectroscopy to determine molecular weight to check peptide identity; b) amino acid analysis to check the identity of the peptide via amino acid composition; c) amino acid sequencing (using an automated protein sequencer or manually) to confirm the defined sequence of amino acid residues; d) HPLC (multiple systems if desired) used to check peptide identity and purity (i.e. identifies peptide impurities); e) water content to determine the water concentration of the peptide compositions; f) ion content to determine the presence of salts in the peptide composition; and g) residual organics to check for the presence of residual organic reagents, starting materials, and/or organic contaminants.

Synthetically produced peptides of the invention comprising up to approximately fifty amino acid residues in length, and most preferably up to approximately thirty amino acid residues in length are particularly desirable as increases in length may result in difficulty in peptide synthesis. Peptides of longer length may be produced by recombinant DNA techniques as discussed below.

Proteins useful in the methods of the present invention may also be produced using recombinant DNA techniques in a host cell transformed with a nucleic acid sequence coding for such peptide. When produced by recombinant techniques, host cells transformed with nucleic acid encoding the desired peptide are cultured in a medium suitable for the cells and isolated peptides can be purified from cell culture medium, host cells, or both using techniques known in the art for purifying peptides and proteins including ion-exchange chromatography, ultra filtration, electrophoresis or immunopurification with antibodies specific for the desired peptide. Proteins produced recombinantly may be isolated and purified to homogeneity, free of cellular material, other polypeptides or culture medium for use in accordance with the methods described above for synthetically produced peptides.

Proteins may also be produced by chemical or enzymatic cleavage of a highly purified full length or native protein of which the sites of chemical digest or enzymatic cleavage have been predetermined and the resulting digest is reproducible. Cleavage can be performed by enzymatic digestion with at least one protease or other suitable enzyme of any living organism. The proteases could be selected among the list according to the Nomenclature Committee of the International Union of Biochemistry and Molecular Biology at http://www.chem.qmw.ac.uk/iumbmb/enzyme/EC34, and the list of the MEROPS database http://www.merops.co.uk and of the Rawlings N D and Barrel A J MEROPS: the peptidase database; Nucl. Acids Res. 28 323-325 (1998), and of Barret A J, Rawlings N D Woessner J F (eds) 1998 Handbook of Proteolytic Enzymes, Academic Press London. Proteins having defined amino acid sequences can be highly purified and isolated free of any other polypeptides or contaminants present in the enzymatic or chemical digest by any of the procedures described above for highly purified, and isolated synthetically or recombinantly produced peptides.

Isolated pure proteins or mixtures containing the proteins according to the present invention may be formulated into pharmaceutical, food or cosmetic compositions of the invention suitable for prophylaxis or therapy in mammals including humans.

Therapeutic or prophylactic compositions of the invention are compositions for oral or parenteral administration or topical application. Preferably, the compositions are administered orally or applied topically.

The pharmaceutical compositions of the inventions may be in the form of conventional pharmaceutical oral dosage forms such as tablets, granules, powders, capsules, gels, pastes, syrups, potions, aerosols, eye drops, or sprays. A pharmaceutical composition may also be incorporated in the filter of a cigarette for binding BaP in cigarette smoke prior to inhaling. Food compositions are usually in the form of conventional functional food products or food supplements, such as candy, other confectionery materials, drinks. Cosmetic compositions are usually in the form of creams, ointments, shampoos, rinses or balms.

In addition to the SAM-dependent methyltransferase or a function-conservative variant or fragment thereof, having a SAM-binding domain specifically binding benzo(a)pyrene, the inventive composition also contains a carrier. The carrier may be a conventional pharmaceutical, food or cosmetic carrier. This carrier may be in any of a variety of forms, such as a powder, a gel, a paste, a tablet, a capsule, a gum, a lozenge, an aerosol, and a fluid. For example, the carrier may be a candy, a chewable gum, or a filter of a cigarette The carrier may include an additive that facilitates its use in an oral cavity, such as a texture-enhancement agent, a chewing-enhancement agent, a thickening agent, and a viscosity-enhancement agent. The carrier may also include flavoring agents, such as sweeteners (sugar, sorbitol, saccharin, or aspartame, etc.), natural or artificial flavors or oils, such as fruit, spice or herbal flavors or oils (cinnamon, mint, or clove oil, etc.), and the like, chlorophyll and/or colorings, such as any suitable conventional coloring agent.

For oral administration, it may be necessary to coat a composition containing the protein of the invention with, or co-administer the composition with, a material to prevent its inactivation or enhance its absorption and bioavailability. For example, a protein formulation may be co-administered with enzyme inhibitors or in liposomes. Enzyme inhibitors include diisopropytfluorophosphate (DEP), pancreatic trypsin inhibitor and trasylol. Liposomes include water-in-oil-in-water CGF emulsions as well as conventional liposomes (cf. Strejan et al., (1984) J. Neuroimmunol., 7:27). When a protein is suitably protected, the protein may be orally administered, for example, with an inert diluent or an assimilable edible carrier. The protein and other ingredients may also be enclosed in a hard or soft shell gelatin capsule, compressed into tablets, or incorporated directly into the individual's diet.

If a therapeutic composition of the invention is to be administered by injection (i.e. subcutaneous injection), then it is preferable that the highly purified protein be soluble in an aqueous solution at a pharmaceutically acceptable pH (i.e. pH range of about 4-9) such that the composition is fluid and easy syringability exists. The composition also preferably includes a pharmaceutically acceptable carrier. As used herein “pharmaceutically acceptable carrier” includes any and all excipients, solvents, dispersion media, coatings, antibacterial and antifungal agents, toxicity agents, buffering agents, absorption delaying or enhancing agents, surfactants, and micelle forming agents, lipids, liposomes, and liquid complex forming agents, stabilizing agents, and the like. The use of such media and agents for pharmaceutically active substance is known in the art. Except insofar as any conventional media or agent is incompatible with the active compound, use thereof in the therapeutic compositions is contemplated. Supplementary active compounds can also be incorporated into the compositions.

Therapeutic compositions of the invention may also be formulated in the form of sterile aqueous solutions prepared by incorporating active compound (i.e., one or more highly purified and isolated protein as described above) in the required amount in an appropriate vehicle with one or a combination of ingredients enumerated above and below, as required, followed by filtered sterilization. Preferred pharmaceutically acceptable carriers include at least one excipient such as sterile water, sodium phosphate, mannitol, sorbitol, or sodium cloride or any combination thereof. Other pharmaceutically acceptable carriers which may be suitable include solvents or dispersion medium containing, for example, water, ethanol, polyol (for example glycerol, propylene glycol, and liquid polyethylene glycol, and the like), suitable mixtures thereof, and vegetable oils. The proper fluidity can be maintained for example by the use of coating such as lecithin, by the maintenance of the required particle size in the case of dispersions and by the use of surfactants. Prevention of the action of microorganisms can be achieved by various antibacterial and antifungal agents, for example, parabens, chlorobutanol, phenol, ascorbic acid, thirmerosol and the like. Prolonged absorption of the injectable compositions can be brought about by including in the composition, an agent which delays absorption, for example, aluminum monostearate and gelatin.

A therapeutic composition of the invention should be sterile, stable under conditions of manufacture, storage, distribution and use and should be preserved against the contaminating action of undesired microorganisms such as bacteria and fungi. A preferred means for manufacturing a therapeutic compositions of the invention in order to maintain the integrity of the composition (i.e. prevent contamination, prolong storage, etc.) is to prepare the formulation of protein and pharmaceutically acceptable carrier(s) such that the composition may be in the form of a lyophilized powder which is reconstituted just prior to use in a pharmaceutically acceptable carrier, such as sterile water. In the case of sterile powders for the preparation of sterile injectable solutions, the preferred methods of preparation are vacuum drying, freeze-drying or spin drying which yields a powder of the active ingredient plus any additional desired ingredient from a previously sterile-filtered solution thereof. Specific formulations of therapeutic compositions of the invention are described below and in the Examples.

In many cases, a therapeutic composition of the invention comprises more than one isolated protein. A therapeutic composition comprising a multi protein formulation suitable for pharmaceutical administration to humans may be desirable for administration of several active proteins. The multi protein formulation includes at least two or more isolated proteins having a defined amino acid sequence. Special considerations when preparing a multi protein formulation include maintaining the solubility, and stability of all proteins in the formulation in an aqueous solution at a physiologically acceptable pH. This requires choosing one or more pharmaceutically acceptable solvents and excipients which are compatible with all the proteins in the multi protein formulation. For example, suitable excipients include sterile water, mannitol, sodium phosphate, or both sodium phosphate and mannitol. An additional consideration in a multi protein formulation is the prevention of dimerization of the proteins, if necessary. Agents may be included in the multi protein formulation which prevent dimerization such as EDTA or any other material or procedures known in the art to prevent dimerization.

In the following, a preferred pharmaceutical composition according to the present invention is given.

| | |
|---|---|
| GNMT: | 0.75 mg protein |
| Buffer: | saline (0.9 % NaCl) |
| Bulking agent: | glycerin |
| Stabilizer | phospholipids (0.1%) |

100 mM phosphate may be used as an alternative buffer. Alternative bulking agents are mannitol and dextrose.

Administration of the therapeutic compositions as described above to an individual can be carried out using known procedures at dosages and for periods of time effective to cause a prevention or treatment of carcinogenesis of the individual.

Effective amounts of the therapeutic compositions of the invention will vary according to factors such as the age, sex, and weight of the individual. A therapeutic composition of the invention may be administered by oral administration, injection (subcutaneous, intravenous, etc.), subligual, inhalation, transdermal application, rectal administration, or any other common route of administration of therapeutic agents. It may be desirable to administer simultaneously or sequentially a therapeutically effective amount of one or more of the therapeutic compositions of the invention to an individual. Each of such compositions for administration simultaneously or sequentially, may comprise only one protein or may comprise a multi protein formulation as described above.

For parenteral administration of one or more compositions of the invention, preferably 0.01 μg-500 mg and more preferably from 0.3 μg-50 mg of each active component (protein) per dosage unit may be administered. For oral administration of one or more compositions of the invention, preferably 0.01 μg-500 mg and more preferably from 0.3 μg-50 mg of each active component (protein) per dosage unit may be administered. It is especially advantageous to formulate parenteral compositions or oral compositions in unit dosage form for ease of administration and uniformity of dosage. Unit dosage form as used herein refers to physically discrete units suited as unitary dosages for human subjects to be treated; each unit containing a predetermined quantity of active protein calculated to produce the desired therapeutic effect in association with the desired pharmaceutical carrier. The specification for the novel unit dosage forms of the invention are dictated by and directly dependent on (a) the unique characteristics of the active compound and the particular therapeutic effect to be achieved, and (b) the limitations inherent in the art of compounding such an active compound for the treatment of human subjects.

Dosage regimen may be adjusted to provide the optimum therapeutic response. For example, several divided doses may be administered over the course of days, weeks, months or years, or the dose may be proportionally increased or reduced with each subsequent injection as indicated by the exigencies of the therapeutic situation. In one preferred therapeutic regimen, subcutaneous injections of therapeutic compositions are given once a week for 1 to 3 weeks. The dosage may remain constant for each administration or may increase or decrease with each subsequent administration.

The invention will now be illustrated by the following non-limiting examples.

### EXAMPLES

### Example 1

### In Vitro Tests

1. Materials and Methods

1.1 Cell lines and culture. Two HCC cell lines-Huh 7 (13) and HA22T/VGH (14)—and one human hepatoblastoma cell line-Hep G2 (15)-were used in this study. Cells were cultured in Dulbecco's modified Eagle's medium (DMEM, GIBCO BRL, Grand Island, N.Y.) with 10% heat-inactivated fetal bovine serum (HyClone, Logan, Utah), penicillin (100 IU/ml), streptomycin (100 μg/ml), nonessential amino acids (0.1 mM), fungizone (2.5 mg/ml) and L-glutamine (2 mM) in a humidified incubator with 5% CO₂.

1.2 Construction of pGNMT, pGNMT-antisense and pGNMT-His-short plasmids. To construct plasmid-pGNMT containing the CMV promoter and GNMT cDNA fragment, we used plasmid-pFLAG-CMV-5 (Kodak, Rochester, N.Y.) as a vector and the pBluescript-GNMT-9-1-2 phagemid (8) as the PCR template for generating the insert. A 0.9 kb DNA fragment containing the GNMT cDNA sequence and restriction enzyme sites on both ends was amplified. All PCR conditions were as recommended by the manufacturer (Perkin Elmer, Norwalk, Conn.) with two exceptions: 2 mM of MgCl₂ and 150 nM of primer. Twenty amplification cycles were performed using Perkin Elmer's Amplitaq Gold Taq DNA polymerase with DNA Thermal Cycler. Each PCR cycle entailed a primer-annealing step at 60° C. for 30 sec and an extension step at 72° C. for 30 sec. The upstream primer (5′-gcggaattcATGGTGGACAGCGTGTAC-3′) included a 3-bp “clamp” (gcg) at the 5′ end followed by a single restriction enzyme site (EcoRI) and the GNMT cDNA sequence. The downstream primer (5′-gcggaattcGTCTGTCCTCTTGAGCAC-3′) contained a similar structural motif as the upstream primer, however, it consisted of a negative strand sequence from the terminal region of the GNMT cDNA. Immediately following amplification, SDS (0.1%) and EDTA (5 mM) were added to the PCR reaction; DNA was precipitated with 2.5 M ammonium acetate and 70% ethanol. After digestion with EcoRI, the DNA fragment was isolated by elution from the agarose gel and ligated to EcoRI-digested pFLAG-CMV-5.

Two primers (F1, 5′-gcggaattcATGGTGGACAGCGTGTAC-3 and R1, 5°-gcggaattcTGTACTCGGCGGTGCGGC-3) were used to construct an antisense-GNMT plasmid (pGNMT-antisense) for amplifying a 136-bp DNA fragment from Phagemid pBluescript-GNMT-9-1-2 (8). The fragment contained an antisense sequence spanning the GNMT translational starting site and two restriction enzyme sites (EcoRI and BamHI) at its terminals. Cloning procedures were similar to those described for pGNMT. To express the GNMT recombinant protein (RP) in E. coli, we constructed a plasmid-pGNMT-His-short. The large S-tag DNA fragment was excised from the pGNMT-His (9) using EcoR I and Nde I restriction enzymes (Stratagene, La Jolla, Calif., USA); the resulting plasmid DNA was re-ligated following a Klenow reaction. Plasmid DNA sequences were confirmed with a DNA sequencer equipped with a dye terminator cycle sequencing core kit (Applied Biosystems Model 373A, Version 1.0.2, Foster City, Calif.).

1.3 GNMT RP expression and purification. pGNMT-His-short was used to transform the E. coli BL21 bacteria used for IPTG induction (induction time, 3 hr; bacterial culture optical density [OD], 0.6-0.7). GNMT RP purification was performed using a Ni²⁺-charged histidine-binding resin column according to manufacturer guidelines (Novagen, Madison, Wis.). RP concentration was measured with a BCA protein assay (Pierce, Rockford, Ill.); purity was tested by running samples on a 12.5% SDS-polyacrylamide mini-gel (Bio-Rad Laboratories, Richmond, Calif.).

1.4 Transfection. All plasmid DNA samples were prepared using Qiagen mega kits (Hilden, Germany). Standard calcium phosphate coprecipitation methodology (16) was used to transfect cultured cells from various liver cancer cell lines with plasmid DNA. Forty-eight hours post-transfection cells were treated with different concentrations (1 to 10 μM) of BaP (Sigma-Aldrich, Steinheim, Germany) dissolved in DMSO (Nacalaitesque, Osaka, Japan) for 16 hrs. Treated cells were subjected to either IFA or ³²p-postlabeling. To produce a negative control, 0.1% DMSO was added to the cell culture.

1.5 Establishing stable clones expressing GNMT. Using calcium phosphate methodology, Hep G2 cells were co-transfected with pGNMT and pTK-Hyg (Clontech, Palo Alto, Calif.) plasmid DNAs. Cells were placed in a selection medium containing hygromycin (300 μg/mL) (17). More than 12 clones were selected and GNMT expression was analyzed with a Western blot assay (WB) using cell lysate collected from each clone. Among them, SCG2 1-1 and 1-11 were chosen for further study based on their expression level of GNMT. SCG2-neg, a stable clone selected from Hep G2 cells co-transfected with pFLAG-CMV-5 and pTK-Hyg plasmids was also used as a control in this study.

1.6 Indirect Immunofluorrescent antibody assay (IFA). Cultured HA22T/VGH or Huh 7 cells were placed on cover slides, treated with 10 μM BaP or 0.1% DMSO, fixed with solution I (4% paraformaldehyde, 400 mM sucrose in PBS) at 37° C. for 30 min, with solution 11 (fixing solution I plus 0.5% Triton X-100) at room temp for 15 min, and with blocking buffer (0.5% BSA in PBS) at room temp for 1 hr. After washing with PBS, the slides were allowed to react with various primary antibodies at 4° C. overnight. The two antibodies were anti-Flag monoclonal (Kodak, Rochester, N.Y.) at 1:500 dilution and rabbit anti-GNMT antiserum-R4 (12) at 1:200 dilution. FITC-conjugated anti-mouse IgG and TRITC-conjugated anti-rabbit IgG (Sigma-Aldrich) were used as secondary antibodies. After 4 washes with PBS, slides were mounted and observed using a confocal fluorescence microscope (TCS-NT, Hilden, Germany). DNA was stained with Hoechst H33258 (Sigma-Aldrich) in order to localize cell nuclei.

1.7 Generating adenovirus-carrying GNMT cDNA (Ad-GNMT). To construct a GMNT recombinant adenovirus controlled by a CMV promoter, pGEX-GNMT (9) was digested with XhoI (filled-in) and Bam HI prior to insertion into the XbaI (filled-in) and BamHI sites of pBluescript SK (−) (Stratagene). GNMT cDNA was also cloned into the HindIII and NotI sites of pAdE1 CMV/pA (18) (a shuttle vector containing the left arm of a virus genome) to generate pXCMV-GNMT. A recombinant adenovirus appeared within 7 to 12 days following the co-transfection of pXCMV-GNMT and pJM17 (18) into 293 cells. Individual virus clones were isolated and identified using PCR with primer sets specific to the adenoviral sequence (18), the insertion flanking regions (18), and the GNMT cDNA (8). Virus titer was determined via the plaque assay method described above (18).

1.8 ³²P-postlabeling and five-dimensional thin-layer chromatography (5D-TLC) for quantifying BPDE-DNA adducts. SCG2 cells and HCC cell lines transiently transfected with pGNMT plasmid DNA for 48 hr were used. DNA was extracted from cells treated with 10 μM BaP or 0.1% DMSO (control) for 16 hr (19) and digested with micrococal endonuclease and spleen phosphodiesterase in succinate buffer (20 mM sodium succinato and 10 mM CaCl₂) for 3 hr at 37° C. The resulting 3′-nucleotides were further extracted with butanol solution twice and labeled with γ-³²p-ATP with T4 kinase in labeling buffer at 38° C. for 30 min. 5D-TLC was used to elucidate labeled DNA adducts (20). Relative adduct level (RAL) was calculated as cpm in adducted nucleotides/(cpm in total nucleotides×dilution).

1.9 Aryl-hydrocarbon hydroxylase (AHH) assay. To measure cytochrome p4501 A1 enzyme activity, approximately 100 μg of cellular homogenates were incubated with reactive solution (100 mM HEPES, 0.4 mM NADPH, 1 mM MgCl₂, and 20 μM BaP) at 37° C. for 10 min. Supernatant protein concentrations were determined using a Bio-Rad protein assay kit (Hercules, Calif.). Reactions were stopped by the addition of acetone; extraction was performed with hexane and 1N NaOH. NaOH fractions were read on a spectrofluorometer (Hitahi Instrument, F4500, Japan) with excitation and emission wavelengths of 396 nm and 522 nm, respectively. Reaction product (3-hydroxy-BaP) concentrations were calculated by comparison with a standard; procedural details are given in (21).

1.10 Western blot (WB) assay. WB was used to detect GNMT in transfected cells or SCG2 clones. Anti-GNMT mAb 14-1 was used to detect GNMT (9). A detailed description of WB procedures is presented in (22).

1.11 Lamarckian genetic algorithm (LGA) dockings. LGA was used to elucidate interaction sites between BaP and various forms of GNMT. Autodock 3.0 software was used to identify the most favorable ligand binding interactions. Van der Waals' hydrogen bonding, hydrophobic desolvations, and electrostatic and torsional free energy were empirically determined to reproduce ligand-protein binding free energies (23). X-ray crystallography data from rat GNMT was used for docking due to its 91% amino acid sequence homology with human GNMT (24, 25). Interactions between BaP and methyltransferase-1 VID (26), 1 HMY (27), 2ADM (28), 1 DCT (29), 1 BOO (30), 2DPM (31). 1 EG2 (32), and 1 G55 (33) were analysed. Parameters were as follows: 10 runs; a population size of 50; and a run-termination criterion of a maximum of 27,000 generations or 2.5×10⁵ energy evaluations, whichever came first. A rmsd conformational clustering tolerance of 0.5 A was calculated from the ligand's crystallographic coordinates. Procedural details are available in (34).

1.12 GNMT enzyme activity assay. GNMT RP purified from a Ni²⁺-charged histidine-binding resin column was used for an enzyme activity assay. GNMT RP (10 mg) was mixed with 10, 50, or 100 μM BaP or DMSO solvent (control) at room temp for 60 min prior to treatment with 100 μL of 100 mM Tris buffer (pH 7.4) containing 50 mM glycine, 0.23 mM SAM, and 2.16 μM S-adenosyl-_{L}-[methyl-3H]-methionine (76.4 Ci/mmol). Following incubation at 37° C. for 30 min, individual reactions were terminated by the addition of a 50 μL mixture of 10% trichloroacetic acid and 5% activated charcoal. Each reaction was performed in triplicate. This procedure has been described in detail by Cook and Wagner (35).

2. Results

2.1 GNMT nuclear translocation was induced by BaP in both HA22T/VGH and Huh 7 cells. GNMT was expressed in the cytoplasm of HA22/VGH cells 48 hr post-transfection with pGNMT DNA (double IFA with both rabbit anti-GNMT antiserum and mouse anti-Flag mAb) (FIGS. 1A and B). Similar results were noted in control Huh 7 cells treated with DMSO solvent (FIGS. 1C and D). In contrast, GNMT proteins were only partly translocated into the nuclei of Huh 7 cells treated with 10 μM BaP for 16 hr (FIGS. 1E, and F). DNA was stained with Hoechst H33258 to localize cell nuclei (FIGS. 1D and F).

2.2 Inhibitory effect of GNMT on BPDE-DNA adduct formation. ³²p-postlabeling and 5-D TLC were used to quantify BPDE-DNA adduct formation. Following treatment with 10M BaP for 16 hr, BPDE-DNA adduct formation in Hep G2, Huh 7, and HA22T/VGH cells transfected with pGNMT decreased 52.8, 13.5 and 20.7% o respectively, compared with cells transfected with the vector plasmid (Table 1). Since the inhibitory effect of GNMT on BPDE-DNA adduct formation was strongest in the Hep G2 cells, we used that cell line as the target in subsequent experiments. Hep G2 cell DNA transfection efficiency was approximately 30%. In addition to pGNMT, a plasmid containing an anti-sense GNMT sequence was constructed for the purpose of verifying the specificity of the GNMT effect. Following BaP treatment, a 34.1% decrease was noted in BPDE adducts formed in pGNMT transfected cells compared with cells transfected with the vector control plasmid (FIG. 8A, lanes 3 and 4). In contrast, a 29.2% increase in BPDE adducts was noted in Hep G2 cells transfected with pGNMT-antisense (lane 5). Quantities of BPDE-DNA adducts formed in cells transfected with equal amounts (20 μg) of pGNMT and pGNMT-antisense were approximately equal to those formed in the vector control cells (lane 6). GNMT expression in different transfection experiments and the effects of antisense GNMT cDNA plasmid construct (pGNMT antisense) were verified by WB assays with mouse anti-GNMT mAb. As shown in lane 4 of FIG. 2B, GNMT was not detected in the lysates of cells transfected with equal amounts of pGNMT and pGNMT antisense.

TABLE 1
Effects of GNMT Expression on BPDE-DNA Adduct Formation
in HCC Cell Lines.

| | BPDE-DNA adducts (RAL) in^{a} | | |
|---|---|---|---|
| ^{b}Cells transfected with | Hep G2 | Huh 7 | HA22TNGH |
| pGNMT | 261.4 (47.2%) | 70.9 (86.5%) | 86.6 (79.3%) |
| pCMV vector | 553.5 (100%) | 82.0 (100%) | 109.1 (100%) |
| no transfection | 625.0 | NT | 161.7 |

a: Relative adducts level (RAL) per 10⁸ nucleotides; measured by v32p-postlabeling method.
b: Transfection efficiency: Hep G2, 30%; Huh 7, 45%; HA22T/VGH, 60%.

Two stable clones (SCG2-1-1 and SCG2-1-11) from Hep G2 cells transfected with pGNMT were used in the same experiments described above. Results from a Northern blot assay indicate that copy numbers (per cell) of GNMT cDNA present in SCG2-1-1 and SCG2-1-11 cells were 3 and 1, respectively (data not shown). Results from a WB assay showed that the GNMT expression level in the SCG2-1-1 cells was nearly three times that in the SCG2-1-11 cells (FIG. 2D, lanes 2 and 3). After treating the SCG2-1-1 and SCG2-1-11 cells with 1 or 10 μM BaP, BPDE-DNA adduct formation inhibition was proportional to GNMT-expression levels under both treatment conditions (FIG. 2C).

The same experiment were carried out using adenovirus-carrying GNMT cDNA (Ad-GNMT). A positive linear relationship was noted between the MOIs of the Ad-GNMT and BPDE-DNA-adduct formation inhibition (FIG. 3). Compared with Ad-GFP-control-infected cells, the Ad-GNMT MOIs increased from 100 to 250 to 1,000 and BPDE-DNA adduct formation decreased 19.5, 36.6, and 61.8%, respectively (FIG. 3A). GNMT-expression levels in Hep G2 cells infected with 100 MOIs of Ad-GFP control, 100, 250, and 1,000 MOIs of Ad-GNMT were analyzed by WB; results are shown in FIG. 3B, lanes 1-4.

2.3 GNMT effect on CYP1A1 enzyme activity induced by BsP, SCG2-1-1 and SCG2-neg cells were treated with varying concentrations of BaP for 16 hr before using AHH assay to measure their cellular CYP1A1 enzyme activity. CYP1A1 activity in cells treated with 3, 6 and 9 μM BaP were 24.5, 41.5 and 71.3 pmol/mg/min for SCG2-neg cells, respectively, and 20.1, 27.7 and 36.2 pmol/mg/min for SCG2-1-1 cells, also respectively (FIG. 4). For cells treated with 9 μM BaP, this represents a 45% reduction in CYP1A1 enzyme activity in GNMT-expressing cells (i.e., SCG2-1-1) compared to SCG2-neg cells.

2.4 Modeling GNMT-BaP interaction. LGA was used to predict physical GNMT-BaP interaction. Again, due to its 91% homology with human GNMT proteins, rat GNMT X-ray crystallography was used for the BaP docking experiments. As shown in FIGS. 5A and 5B, we found that BaP binds with both dimeric (yellow) and tetrameric (cyan) forms of GNMT, but that it prefers binding with the dimeric form (protein databank PDB code; 1D2C). This cluster is located at the intersection of the SAM- and SAH-binding sites (Table 2 and FIG. 5B). The low (−9.10 Kcal/mole) binding energy between the dimeric form of GNMT and BaP suggests that BaP may displace the SAM position; the high (254.9 Kcal/mole) binding energy of BaP with a GNMT dimer already bound with SAM (PDB code: 1 XVA) suggests that BaP and SAM are in competition for binding with GNMT (Table 2). Accordingly, several GNMT amino acid residues (including Thr37, Gly137 and His142 of one dimer subunit and Glu15 of another subunit) are in close proximity to BaP (FIG. 5C).

TABLE 2
Lamarckian Genetic Algorithm Dockings of GNMT Protein and BaP Molecules.

| PDB | Small | Cluster | Cluster | Mean Energy | Number of | Protein |
|---|---|---|---|---|---|---|
| code^{a} | molecule | number | population | (Kcal/mol) | evaluations | details |
| 1D2H^{b} | BaP | 3 | 5 | −3.22 | 2.5 × 10⁵ | R175K + SAH |
| | | | | | | Tetramer |
| 1XVA^{c} | BaP | 5 | 5 | +254.9 | 2.5 × 10⁵ | +SAM |
| | | | | | | Dimer |
| 1XVA^{c} | SAM | 2 | 5 | −9.85 | 2.5 × 10⁵ | −SAM |
| | | | | | | Dimer |

^{a}PDB: protein data bank (http://www.resb.org/pdb).
^{b}Cluster is located at the intersection of SAM and SAH.
^{c}Bad is −2 A from SAM; the high energy level suggests that such a complex is difficult to form.
^{d}BaP displaces the SAM position.
^{e}RMSD = 2.70 A. A second cluster (n = 5) corresponds to the known crystal structure at an RMSD of 0.68 A and a mean energy of −8.80 Kcal/mol. Note the nearby location of an acetate ion that might serve to stabilize the second cluster.

2.4 BaP-induced inhibition of GNMT enzyme activity. Based on the inference that BaP can bind with GNMT, the potential effects of BaP on GNMT enzyme activity was studied by constructing a plasmid-pGNMT-His-Short to express a His-tag-GNMT RP in E. coli. GNMT RP purified from a Ni²⁺-charged histidine-binding resin column was used for our analysis. As shown in FIG. 6, GNMT enzyme activity from reactions containing 10 and 50 μM BaP decreased 44% and 62%, respectively, compared with the DMSO control.

IFA to demonstrate the power of BaP to induce the nuclear translocation of GNMT. Our results show that GNMT not only inhibits BPDE-DNA adduct formation, but also down-regulates CYP1A1 enzyme activity; conversely, BaP also inhibits GNMT enzyme activity. Finally, we used a docking experiment to show the exact location of BaP-GNMT interaction. These results represent a novel finding of a cellular defense mechanism against potentially damaging forms of exposure. We confirmed the inhibition of BPDE-DNA adduct formation by GNMT via transient transfection, stable-clone selection, and adenovirus infection systems, with consistent results throughout. An anti-sense construct for GNMT cDNA was used to demonstrate interaction specifcity (FIG. 2A), and WB assays were used to monitor GNMT expression levels in various gene-transfer experiment sets. The dose-dependent inhibitory effect of GNMT on BPDE-DNA adduct formation was further elaborated with Hep G2 stable clones and a recombinant adenovirus carrying GNMT cDNA (FIGS. 2C and 3A).

Many PAHs induce cytochrome P-450 expression through an aryl hydrocarbon receptor (AhR)-dependent pathway (37). After diffusing into a cell, BaP binds with AhR and translocates into the nuclei, where BaP-AhR heterodimers form complexes with Ah receptor nuclear translocator (Arnt) proteins (2). The BaP-AhR-Arnt complexes then transactivate the CYP1A1 gene via interaction with its xenobiotic-responsive element in the promoter region (38). In addition to the inhibition of BPDE-DNA adduct formation, our results show that GNMT is capable of reducing CYP1A1 enzyme activity induced by BaP (FIG. 4). Foussat et al. (39) used AhR-deficient transgenic mice to demonstrate that GNMT is not a transcriptional activator of the CYP1A1 gene (39). Preliminary data from our real-time PCR analysis showed that following BaP treatment, CYP1A1 gene expression was reduced by approximately 20% in SCG2-1-1 cells compared to Hep G2 cells (manuscript in preparation).

Previous research has shown that the tetrameric form of rat GNMT acts as an enzyme and that the dimeric form of rat GNMT is capable of binding with PAHs (40). In the present invention, LGA and a scoring function was used for estimating binding-related free energy change to locate possible sites for interactions between BaP and various forms of GNMT; X-ray crystallography data for rat GNMT was used for this purpose. The results indicate that a) the BaP-binding domain is located at the substrate (SAM)-binding site of GNMT and b) BaP prefers binding with the dimeric form of GNMT. The R175K mutant form of the GNMT tetramer (PDB code; 1D2G) was used to demonstrate that although R/K residue is near the binding site (−5 A from the SAM position), it exerts practically no effect on GNMT-BaP cluster formation (Table 2). In comparison, the presence of an acetate ion favors the formation of the second preferred cluster in GNMT-SAM binding in the 1XVA crystal structure (Table 2, final entry). It has been demonstrated that of various search systems, the LGA method is the most likely to locate crystallographic structures (23). Heavily populated clusters usually correspond to crystallographically determined positions that show 0.2-0.8 A RMS differences from the crystal structures. For most ligands, our docking simulation predicted single binding modes that matched crystallographic binding modes within 1.0 A RMSD (23). It is shown that LGA is a reliable method for predicting the bound conformation of a ligand to its macromolecular target. BaP-GNMT interaction was also confirmed by a functional assay showing that GNMT enzyme activity was reduced nearly 50% in the presence of BaP (FIG. 6).

Since BaP prefers binding with the SAM-binding domain of GNMT, LGA was used to study interactions between BaP and eight other SAM-dependent methyltransferases (MTases): catechol O-methyltransferase (COMT), HhaI DNA MTase, TaqI DNA MTase, HaeIII DNA MTase, PvuII DNA MTase, DpnII DNA MTase, RsrI DNA MTase, and DNMT2. Our results show that BaP was capable of binding with the HhaI-, HaeIII-, PvuII-DNA MTases and DNMT2 but not with the COMT, TaqI-, DpnII-, and RsrI-DNA MTases (Table 3). It was found that the target atom of all the BaP-preferred DNA MTases is cytosine and not adenine (41). This is the first evidence suggesting that an environmental carcinogen such as BaP has the potential to interact with different DNA MTases. In light of evidence showing that the induction of GNMT enzyme activity by all-trans-retinoic-acid causes DNA hypomethylation in rat hepatocytes (42), it is shown that BaP may affect DNA methylation via interactions with DNMT and GNMT, and thus contribute to a carcinogenic pathway.

TABLE 3
Lamarckian Genetic Algorithm Dockings of Some SAM-dependent
Methylfiransferases and BaP Molecules.^{a}

| PDB | Small | Number of | Cluster | Mean Energy | Number of | Protein |
|---|---|---|---|---|---|---|
| code^{b} | molecule | clusters | population | Kcal/mol | evaluations | details |
| 1VID^{c} | BaP | 2 | 4 | −2.18 | 2.5 × 10⁵ | COMT |
| | | | | | | Monomer |
| 2ADM^{c} | BaP | 4 | 6 | =47.19 | 2.5 × 10⁵ | TaqI |
| | | | | | | DNA-MT |
| | | | | | | Dimer |
| 2DPM^{h} | BaP | 4 | 5 | =13.46 | 2.5 × 10⁵ | DpnII |
| | | | | | | DNA-MT |
| | | | | | | Monomer |
| 1EG2ⁱ | BaP | 4 | 2 | =85.64 | 2.5 × 10⁵ | RsrI |
| | | | | | | DNA-MT |
| | | | | | | Monomer |

^{a}The SAM molecules were removed from the 1 VID, 1HMY. 2ADM and 2DPMmethyltransferase macromolecules before docking. The BaP molecule tried to move into the former SAM position. The SAH molecules were removed from the 1BOO and 1 G55 methyltransferase macromolecules before docking.
^{b}PDB: protein data bank (http://www.resb.org/pdb).
^{c}The energy of the second cluster (population 6/10) was −0.32 Kcal/mol; COMT did not bind with BaP at one preferred position.
^{d}The energy of the second cluster (population 1/10) was −6.45 Kcal/mol; Hhal-DNA-MT bound with BaP at a lower energy-preferred position.
^{e}The high binding energy (+47.19 Kcal/mol) suggests that TaqI DNA-MT does not bind with BaP.
^{f}The energy of the second cluster (population 1/10) was −9.50 Kcal/mol, very close to the lowest energy cluster (population 8/10, energy −9.69 Kcal/mol); therefore, HaeIII DNA-MT bound strongly with BaP at a preferred position.
^{g}The high binding energy (−8.69 Kcal/mol) suggests that PvuII binds with BaP. The binding energies of the other two observed clusters (−8.63 Kcal/mol and −8.58 Kcal/mol) were very close to the lowest energy cluster.
^{h}The +13.46 Kcal/mol binding energy suggests that DpnII DNA-MT does not bind with BaP.
ⁱThe +85.64 Kcal/mol binding energy suggests that RsrI DNA-MT does not bind with BaP.
^{j}The −8.70 Kcal/mol binding energy suggests that DNMT2 binds strongly with BaP in a preferred position.

3. Summary

Glycine N-methyltransferase (GNMT) affects genetic stability by (a) regulating the ratio of S-adenosylmethionine (SAM) to S-adenosylhomocystine and (b) binding to folate. Based on the identification of GNMT as a 4S polyaromatic hydrocarbon-binding protein, liver cancer cell lines were used that expressed GNMT either transiently or stably in cDNA transfections to analyze GNMT's role in the benzo[a]pyrene (BaP) detoxification pathway. Results from an indirect immunofluorescent antibody assay show that GNMT was expressed in cell cytoplasm prior to BaP treatment and translocated to cell nuclei following BaP treatment. Compared to cells transfected with the vector plasmid, the number of BPDE-DNA adducts that formed in GNMT expressing cells was significantly reduced. Furthermore, the dose-dependent inhibition of BPDE-DNA adduct formation by GNMT was observed in Hep G2 cells infected with different MOIs of recombinant adenoviruses carrying GNMT cDNA. According to an AHH enzyme activity assay, GNMT inhibited BaP-induced CYP1A1 enzyme activity. Automated BaP docking using a Lamarckian genetic algorithm with GNMT X-ray crystallography revealed a BaP preference for the SAM-binding domain of the dimeric form of GNMT-a novel finding of a cellular defense against potentially damaging exposures. In addition to GNMT, results from docking experiments showed that BaP readily binds with other DNA methyltransferases (MTases), including HhaI-, HaeIII-, PvuII-MTases and human DNMT2. Therefore, BaP-DNMT and BaP-GNMT interactions were shown to contribute to carcinogenesis.

### Example 2

### BaP Binding and Prevention of Carcinogenisis In Vivo

1. GNMT Transgenic Mouse Model for Test

1.1 pPEPCKex-flGNMT Plasmid Construction: pPEPCKex-flGNMT plasmid was prepared by using a pPEPCKex vector (concluding with phosphoenolpyruvate carboxykinase promoter (PEPCK, Valera et al., 1994), specific expressed in liver and kidney) and pSK-flGNMT (concluding with full length human GNMT cDNA) plasmid. Both plasmids were digested with Not I and Xho I. Insert was ligated to the vector and transformation into the competent cell (JM109). The clones were selected with ampicillin and screened with PCR to check pPEPCKex-flGNMT plasmid (FIG. 1).

1.2 Production of Transgenic Mice: pPEPCKex-flGNMT plasmid was amplified and digested with Asc I to linear form (4.3 Kb). The linear form pPEPCKex-flGNMT gene was sent into FVB stain mice 0.5 days embryo by pronuclei microinjection. The embryos were sent into the foster mother (ICR strain mice). After 18˜21 days, the mice were bred and screened with PCR to check the transgenic mice.

1.3 Expression human GNMT in liver and kidney of transgenic mice: To check the PEPCK promoter specific expression organ, we used northern blot (FIG. 2) and western blot (FIG. 3). The human GNMT was specific expression in liver and kidney. GNMT expression level of transgenic mice was higher than normal mice.

1.4 B(a)P Treat on GNMT Transgenic Mice and HBV-largeS Transgenic Mice: Treat with 375 μg B(a)P/7 g body weight everyday by IP injection for 15 days on following 2 groups mice.
1. GNMT transgenic mice
2. Normal mice
   Pathology of the lung of the 2 groups treated with BaP and sacrificed 78 weeks after the challenge (FIG. 4).
      2. Results and Conclusions:
   When GNMT is overexpressed in transgenic mice treated with B(a)P, only 30% of the mice generated lung tumors. Normal mice (no GNMT overexpression) treated with B(a)P, generated a lung tumor at a rate of 66.66%. Accordingly, GNMT can bind B(a)P in vivo and is therefore capable of preventing carcinogenesis.

The abbreviations used are:

GNMT, glycine N-methyltransferase; HCC, hepatocellular carcinoma; PAH, polycyclic aromatic hydrocarbon; BaP, benzo[a]pyrene; BPDE, BaP-7,8-diol 9,10-epoxide; MOI, multiplicity of infection; IPTG, isopropyl-beta-D-thiogalactopyranosid; CYP1A1, cytochrome P4501A1; AhR, aryl hydrocarbon receptor; Amt, Ah receptor nuclear translocator; XRE, xenobiotic-responsive elements; PCR, polymerase chain reaction; AHH, aryl hydrocarbon hydroxylase; PBS, phosphate buffered saline; IFA, indirect immunofluorescent antibody assay; LGA, Lamarckian genetic algorithm; PDB, Protein Data Bank; MTases, Methyltransferases; DNMT2, DNA methyltransferase 2; RAL, relative adducts level.

### REFERENCES

1. Fischman, M. L., Cadman, E. C., and Desmond, S. Occupational Cancer. In LaDou J. edited, Occupational Medicine. p.p. 182-208. Pretince-Hall International, Inc. Conn., USA, 1990.
2. Whitlock, J. P. Jr., Okino, S. T., Dong, L., Ko, H. P., Clarke-Katzenberg, R., Ma, Q., and Li, H. Cytochromes P450 5: Induction of cytochrome P4501A1: a model for analyzing mammalian gene transcription. FASEB J., 10: 809, 818, 1996.
3. Foldes, R. L., Hines, R. N., Ho, K. L., Shen, M. L., Nagel, K. B., and Bresnick, E. 3-Methylchlanthrene-induced expression of the cytochrome P-450c gene. Arch. Biochem. Biophys., 239: 137-146, 1985.
4. Raval, P., Iversen, P. L., and Bresnick, E. Induction of cytochromes P4501A1 and P4501A2 as determined by solution hybridization. Biochem. Pharmacol., 41: 1719-1723, 1991.
5. Wijnhoven, S. W., Kool, H. J., van Oostrom, C. T., Beems, R. B., Mullenders, L. H., van Zeeland, A. A., van der Horst, G. T., Vrieling, H., and van Steeg, H. The relationship between benzo[a]pyrene-induced mutagenesis and carcinogenesis in repair-deficient Cockayne syndrome group B mice. Cancer Res., 60: 5681-5687, 2000.
6. Kerr, S. J. Competing methyltransferase system. J Biol. Chem., 247: 4248-4252, 1972.
7. Yeo, E. J., and Wagner, C. Tissue distribution of glycine N-methyltransferase, a major folate-binding protein of liver. Proc. Natl. Acad. Sci. USA, 91: 210-214, 1994.
8. Chen, Y. M. A., Shiu, J. Y., Tzeng, S. J., Shih, L. S., Chen, Y. J., Lui, W. Y., and Chen, P. H. Characterization of glycine-N-methyltransferase-gene expression in human hepatocellular carcinoma. Int. J. Cancer, 75: 787-793, 1998.
9. Liu, H. H., Chen, K. H., Lui, W. Y., Wong, F. W., and Chen, Y. M. A. Characterization of reduced expression of glycine N-methyltransferase in the cancerous hepatic tissues using two newly developed monoclonal antibodies. J. Biomed. Sci., 10: 87-97, 2003.
10. Chen, Y. M. A., Chen, L. Y., Wong, F. H., Lee, C. M., Chang, T. J., and Yang-Feng, T. L. Genomic structure, expression and chromosomal localization of the human glycine N-methyltransferase gene. Genomics, 66:43-47, 2000.
11. Tseng, T. L., Shih, Y. P., Huang, Y. C., Wang, C. K., Chen, P. H., Chang, J. G., Yeh, K. T., Struewing, J. P., Chen, Y. M. A., and Buetow, K. H. Genotypic and phenotypic characterization of a putative tumor susceptibility gene, GNMT, in liver cancer. Cancer Res., 63: 647-654, 2003.
12. Raha, A., Wagner, C., MacDonald, R. G., and Bresnick, E. Rat liver cytosolic 4S polycyclic aromatic hydrocarbon-binding protein is glycine N-methyltransferase. J. Biol. Chem., 269: 5750-5756, 1994
13. Nakabayashi, H., Taketa, K., Miyano, K., Yamane, T., and Sato, J. Growth of human hepatoma cells lines with differentiated functions in chemically defined medium, Cancer Res., 42: 3858-3863, 1982.
14. Fogh, J., Trempe, G., and Loveless, J. D. New human tumor cell lines in Fogh J. (cd), Human tumor cell in vitro, p. p. 115-119, Plenum, New York, 1977.
15. Aden, D. P., Fogel, A., Plotkin, S., Damjanov, I., and Knowles, B. B. Controlled synthesis of HBsAg in a differentiated human liver-carcinoma-derived cell line. Nature (Lond.), 282: 615-616, 1979.
16. Clark, S. J., Harrison, J., Paul, C. L., and Frommer, M. High sensitivity mapping of methylated cytosines. Nucleic Acids Res., 22: 2990-2997, 1994.
17. Gurtu, V., Yan, G., and Zhang, G. IRES bicistronic expression vectors for efficient creation of stable mammalian cell lines. Biochem. Biophys. Res. Commun., 229: 295-298, 1996.
18. Kleinerman, D., Zhang, W. W., von Eschenbach, A. C., Lin S. H., and Hsieh, J. T. Application of a tumor suppressor gene, C-CAM1, inandrogen-independent prostate cancer therapy: a preclinical study. Cancer Res., 55: 2831-2836, 1995.
19. Vesselinovitch, S. D., Koka, M., Mihailovich, N., and Rao, K. V. N. Carcinogenicity of diethyl-nitrosamine in newborn, infant and adult mice. J. Cancer Res. Clin. Oncol., 108: 60-65, 1984.
20. Roggeband, R., Wolterbeek, A. P., Rutten, A. A., and Baan, R. A. Comparative 32p-postlabeling analysis of benzo[a]pyrene-DNA adducts formed in vitro upon activation of benzo[a]pyrene by human, rabbit and rodent liver microsomes. Carcinogenesis, 14:1945-1950, 1993.
21. Chang, K. W., Lee, H., Wang, H. J., Chen, S. Y., and Lin, P. Differential response to benzo[A]pyrene in human lung adenocarcinoma cell lines: the absence of aryl hydrocarbon receptor activation. Life Sci., 65: 1339-1349, 1999.
22. Rowling, M. J., McMullen, M. H., and Schalinske, K. L. Vitamin A and its derivatives induce hepatic glycine N-methyltransferase and hypomethylation of DNA in rats. J. Nutr., 132: 365-369, 2002.
23. Rosenfeld, R. J., Goodsell, D. S., Musah, R. A., Morris, G. M., Goodin, D. B., and Olson, A. J. Automated docking of ligands to an artificial active site: augmenting crystallographic analysis with computer nnodeling. J. Comput. Aid. Mol. De., 17: 525-536, 2003.
24. Fu, Z., Hu, Y., Konishi, K., Takata, Y., Ogawa, H., Gomi, T., Fujioka, M., and Takusagawa, F. Crystal structure of glycine N-methyltransferase from rat liver. Biochemistry, 35: 11985-11993, 1996.
25. Huang, Y., Komoto, J., Konishi, K., Takata, Y., Ogawa, H., Gomi, T., Fujioka, M., and Takusagawa, F. Mechanisms for auto-inhibition and forced product release in glycine N-methyl transferase: crystal structures of wild-type, mutant R175K and S-adenosylhomocysteine-bound R175K enzymes. J. Mol. Biol., 298: 149-162, 2000.
26. Vidgren, J., Svensson, L. A., arid Liljas, A. Crystal structure of catechol O-methyltransferase. Nature (Lond.), 368: 354-358, 1994.
27. Cheng, X., Kumar, S., Posfai, J., Pflugrath, J. W., and Roberts, R. J. Crystal structure of the HhaI DNA methyltransferase complexed with S-adenosyl-L-methionine. Cell, 74: 299-307, 1993.
28. Schluckebier, G., Kozak, M., Bleimling, N., Weinhold, E., and Saenger, W. Differential binding of S-adenosylmethionine, S-adenosylhomocysteine and Sinefungin to the adenine-specific DNA methyltransferase M. TaqI. J. Mol. Biol., 265:56-67, 1997.
29. Reinisch, K. M., Chen, L., Verdine, G. L., and Lipscomb, W. N. The crystal structure of HaeIII methyltransferase convalently complexed to DNA: an extrahelical cytosine and rearranged base pairing. Cell, 82: 143-153. 1995.
30. Gong, W., O'Gara, M., Blumenthal, R. M., and Cheng, X. Structure of pvuII DNA-(cytosine N4) methyltransferase, an example of domain permutation and protein fold assignment. Nucleic Acids Res., 25: 2702-2715, 1997.
31. Tran, P. H., Korszun, Z. R., Cerritelli, S., Springhorn, S. S., and Lacks, S. A. Crystal structure of the DpnII DNA adenine methyltransferase from the DpnII restriction system of streptococcus pneumoniae bound to S-Adenosylmethionine. Structure, 6: 1563-1575, 1998.
32. Scavetta, R. D., Thomas, C. B., Walsh, M., Szegedi, S., Joachimiak, A., Gumport, R. I., and Churchill, M. E. A. Structure of RsrI methyltransferase, a member of the N6-adenine B class of DNA methyltransferases. Nucleic Acids Res., 28: 3950-3961, 2000.
33. Dong, A., Yoder, J. A., Zhang, X., Zhou, L., Bestor, T. H., and Cheng, X. Structure of human DNMT 2, an enigmatic DNA methyltransferase homolog that displays denaturant-resistant binding to DNA. Nucleic Acids Res., 29: 439-448, 2001.
34. Morris, G. M., Goodsell, D. S., Halliday, R. S., Huey, R., Hart, W. E., Belew, R. K., and Olson, A. J. Automated docking using a lamarckian genetic algorithm and an empirical binding free energy function. J. Comput. Chem., 19: 1639-1662, 1998.
35. Cook, R. J., and Wagner, C. Glycine N-methyltransferase is a folate binding protein of rat liver cytosol. Proc. Natl. Acad. Sci. USA, 81: 3631-3634, 1984.
36. Aida, K., Tawata, M., Negishi, M., and Onaya, T. Mouse glycine N-methyltransferase is sexually dimorphic and regulation by growth hormone. Horm. Metab. Res, 29: 646-649, 1997.
37. Okey, A. B., Bendy, G. P., Mason, M. E., Nebert, D. W., Forster-Gibson, C. J., Munean, land Dufresne, M. J. Temperature-dependent cytosol-to-nucleus translocation of the Ah receptor for 2,3,7,8-tetrachlorodibenzo-p-dioxin in continuous cell culture lines. J. Biol. Chem., 255: 11415-11422, 1980.
38. Hapgood. J., Cuthill, S., Denis, M., Poellinger, L., and Gustafsson, J. A. Specific protein-DNA interactions at a xenobiotic-responsive element: copurification of dioxin receptor and DNA-binding activity. Proc. Natl. Acad. Sci, USA, 86: 60-64, 1989.
39. Foussat, J., Costet, P., Gaitier, P., Pineau, T., and Lesca, P. The 4S benzo(a)pyrene-binding protein is not a transcriptional activator of CYP1A1 gene in Ah receptor-deficient (AHR −/−) transgenic mice. Arch. Bioehem. Biophys., 349: 349-355, 1998.
40. Bhat, R., Wagner, C., and Bresnick, E. The homodimic form of glycine N-methyl transferase acts as a polycyclic aromatic hydrocarbon-binding receptor. Biochem. J., 36: 9906-9910, 1997.
41. Chang, X., and Roberts, R. J. AdoMet-dependent methylation. DNA methyltransferases and base flipping. Nucleic Acids Res., 29: 3784-3795, 2001.
42. Rowling, M. J., McMullen, M. H., and Schalinske, K. L. Vitamine A and its derivatives induce hepatic glycine N-methyltransferase and hypomethylation of DNA in Rats. J. Nutr., 132: 365-369, 2002.

The article “a” and “an” are used herein to refer to one or more than one (i.e., to at least one) of the grammatical object of the article. By way of example, “an element” means one or more element.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. A pharmaceutical, food or cosmetic composition comprising a carrier and an effective amount of an active benzo(a)pyrene binding protein, whereby the protein is a SAM-dependent methyltransferase or a function-conservative variant or fragment thereof, having a SAM-binding domain specifically binding benzo(a)pyrene.

2. The pharmaceutical, food or cosmetic composition according to claim 1, wherein said pharmaceutical or food composition is adequate for oral or parenteral administration.

3. The pharmaceutical, food or cosmetic composition according to claim 1, wherein the methyltransferase is selected from the group of GNMT, HhaI-DNA MTases, HaeIII-DNA MTases, and PvuII-DNA MTases.

4. The pharmaceutical, food or cosmetic composition according to claim 2, wherein the methyltransferase is selected from the group of GNMT, HhaI-DNA MTases, HaeIII-DNA MTases, and PvuII-DNA MTases.

5. The pharmaceutical, food or cosmetic composition according to claim 3, wherein the methyltransferase is GNMT.

6. The pharmaceutical, food or cosmetic composition according to claim 4, wherein the methyltransferase is GNMT.

7. The pharmaceutical, food or cosmetic composition according to claim 1 wherein the function-conservative variant or fragment of the SAM-dependent methyltransferase comprises the amino acid sequence of SEQ ID NO:1.

8. The pharmaceutical, food or cosmetic composition according to claim 1, which is a microorganism or a mixture extracted from a microorganism or an organ of an animal.

9. Use of SAM-dependent methyltransferase or a function-conservative variant or fragment thereof, having a SAM-binding domain specifically binding benzo(a)pyrene, for the manufacture of a medicament for the prevent or treatment of cancer.

10. The use according to claim 9, wherein the cancer is hepatoma, lung cancer, bladder cancer, prostate cancer, colon cancer, brain tumor, breast cancer, and kidney cancer of mammals including humans.

11. A method for the prevention or treatment of cancer which comprises administering a pharmaceutically effective amount of a SAM-dependent methyltransferase or a function-conservative variant or fragment thereof, having a SAM-binding domain specifically binding benzo(a)pyrene to an individual.

12. The method according to claim 11, wherein the individual is a human.


SEQUENCE LISTING (1 sequence)

SEQ ID NO: 1 - protein, 295 aa, Homo sapiens
MVDSVYRTRSLGVAAEGLPDQYADGEAARVWQLYIGDTRSRTAEYKAWLLGLLRQHGCQRVLDVACGTGVDSIMLVEEGFSVTSVDASDKMLKYALKERWNRRHEPAFDKWVIEEANWMTLDKDVPQSAEGGFDAVICLGNSFAHLPDCKGDQSEHRLALKNIASMVRAGGLLVIDHRNYDHILSTGCAPPGKNIYYKSDLTKDVTTSVLIVNNKAHMVTLDYTVQVPGAGQDGSPGLSKFRLSYYPHCLASFTELLQAAFGGKCQHSVLGDFKPYKPGQTYIPCYFIHVLKRTD